(19) United States
(12) Patent Application Publication
(10) Pub. No.: US 2011/0262534 A1
(43) Pub. Date: Oct. 27, 2011

(54) POLYSACCHARIDE CAPSULE ENCLOSING A FATTY ACID OIL-CONTAINING EMULSION

(76) Inventors: Gunnar Berge, Oslo (NO); Svein Olaf Hustvedt, Oslo (NO); Thomas Andersen, Oslo (NO); Olav Gåserød, Steinberg (NO); Peder Oscar Andersen, Oslo (NO); Christian Klein Larsen, Lillestrom (NO)

(21) Appl. No.: 13/062,997

(22) PCT Filed: Sep. 10, 2009
(86) PCT No.: PCT/IB2009/006933

§ 371 Date: Jun. 24, 2011

Related U.S. Application Data
(63) Continuation of application No. 12/207,824, filed on Sep. 10, 2008.

(51) Int. Cl. A61K 9/48; A61K 31/232; A61P 3/06; A61P 9/00; A61P 9/10; A61P 37/02; A61P 3/08; A61P 25/00; A61P 9/04; A61K 31/202; A61P 27/02

(52) U.S. Cl. 424/455; 514/560; 514/549

(57) ABSTRACT

Novel capsules where an outer shell comprises a polysaccharide, e.g. an alginate. In the capsules there is an emulsion comprising a fatty acid oil mixture and at least one surfactant. Preferred fatty acid oils are eicosapentaenoic acid (EPA) and doccosahexaenoic acid (DHA).

This application claims priority to U.S. application Ser. No. 12/207,824, filed on Sep. 10, 2008, which is incorporated herein by reference in its entirety.

New capsules comprising at least one oily phase that comprises a fatty acid oil mixture and at least one surfactant in an alginate capsule formulation, methods of preparing the same, and uses thereof are disclosed herein.

Compositions comprising at least one oily phase comprising a fatty acid oil mixture encapsulated in an alginate outer surface shell are disclosed. The compositions may be seamless capsules with a shell that is thinner compared to gelatin capsules, thereby allowing a larger amount of material to be encapsulated. The compositions, i.e., capsules, of the present disclosure may thus be administered to a subject for therapeutic treatment and/or regulation of at least one health problem including, for example, irregular plasma lipid levels, cardiovascular functions, immune functions, visual functions, insulin action, neuronal development, hypertriglyceridemia, heart failure, and post myocardial infarction (MI).

In humans, cholesterol and triglycerides are part of lipoprotein complexes in the bloodstream and can be separated via ultracentrifugation into high-density lipoprotein (HDL), intermediate-density lipoprotein (IDL), low-density lipoprotein (LDL), and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C and VLDL-C) promote human atherosclerosis and decreased levels of HDL-C and its transport complex, apolipoprotein A, which are associated with the development of atherosclerosis. Furthermore, cardiovascular morbidity and mortality in humans can vary directly with the level of total-C and LDL-C and inversely with the level of HDL-C. In addition, researchers have found that non-HDL cholesterol is an important indicator of hypertriglyceridemia, vascular disease, atherosclerotic disease, and related conditions. In fact, recently non-HDL cholesterol reduction has been specified as a treatment objective in NCEP ATP III.

Omega-3 fatty acids may regulate plasma lipid levels, cardiovascular and immune functions, insulin action, and neuronal development, and visual function. Marine oils, also commonly referred to as fish oils, are a source of omega-3 fatty acids, including eicosapentaenoic acid (EPA) and docosahexaenoic acid (DHA), that have been found to regulate lipid metabolism. Plant-based oils and microbial oils are also sources of omega-3 fatty acids. Omega-3 fatty acids may have beneficial effects on the risk factors for cardiovascular diseases, for example hypertension and hypertriglyceridemia, and on the coagulation factor VII phospholipid complex activity. Omega-3 fatty acids may also lower serum triglycerides, increase serum HDL cholesterol, lower systolic and diastolic blood pressure and/or pulse rate, and may lower the activity of the blood coagulation factor VII-phospholipid complex. Further, omega-3 fatty acids are generally well-tolerated, without giving rise to severe side effects.

One form of omega-3 fatty acid is a concentrate of omega-3, long chain, polyunsaturated fatty acids from fish oil containing DHA and EPA, such as sold under the trademark Omacor®/Lovaza™/Zodinφ/Seacor®. See, for example, U.S. Pat. Nos. 5,502,077, 5,656,667 and 5,698,594. In particular, each 1000 mg capsule of Lovaza™ contains at least 90% omega-3 ethyl ester fatty acids (84% EPA/DHA); approximately 465 mg EPA (eicosapentaenoic acid) ethyl ester and approximately 375 mg DHA (docosahexaenoic acid) ethyl ester.

The formulation of drugs into capsules, for example, soft or hard gelatin capsules, has been reported to solve problems associated with tablets. Stability has generally improved through the use of gelatin capsules, most notably with active pharmaceutical ingredients (APIs) that may be susceptible to oxidation and hydrolysis. An example is vitamin A which is relatively unstable in air and light; however, when encapsulated, the contents show no significant loss of potency for 3 years or longer when stored and packaged under prescribed conditions of temperature and humidity. U.S. Patent Application Publication No. 2004/0224020 discloses an oral dosage form with active agents in controlled cores and in immediate release gelatin capsule coats.

Alginate capsule formulations have been reported. For example, FR 2 745 979 discloses alginate capsules comprising omega-3 fatty acids as animal feed additives. Further, for example, HU 2 030 38 discloses encapsulation of unsaturated fatty acids, fatty acid esters, and their mixtures using alginated gel.

Several references disclose enteric capsules containing omega-3 fatty acids. For example, U.S. Pat. No. 6,531,150 discloses enteric capsules having a buffer layer of a water-soluble gel containing an acid or acid salt between the content of omega-3 fatty acids and the gelatin-based coating layer. Further, for example, European Patent Application No. EP 1529524 and German Application No. DE 19930030 disclose gelatin capsules containing omega-3 fatty acids coated with xylose to provide resistance to gastric juice and increase stability. In addition, Belluzi et al., N. Eng. J. Med., 334(24):1557-60, 1996, and Belluzi et al., Gastroenterology, 102(4) pt. 2: A542, 1992, each disclose enteric coated fish oil capsules (PUREPA® Tillotts-Pharma) for delayed delivery.

It is to be understood that both the foregoing general description and the following detailed description are exemplary and explanatory only and are not restrictive of the invention, as claimed.

The present disclosure is further directed to a capsule comprising a polysaccharide gel membrane outer surface shell comprising at least one alginate wherein: the outer surface encapsulates an emulsion comprising at least one oily phase; the at least one oily phase comprises a fatty acid oil mixture and at least one surfactant; the fatty acid oil mixture comprises at least 75% eicosapentaenoic acid (EPA) and docosahexaenoic acid (DHA), by weight of the fatty acid oil mixture; and the emulsion does not comprise marmelo mucilage.

The present disclosure is further directed to an oil-in-water emulsion to be encapsulated comprising: from about 80% to about 85% of at least one fatty acid oil mixture by weight of the emulsion; wherein the fatty acid oil mixture comprises at least 90% omega-3 ethyl ester fatty acids, by weight of the fatty acid oil mixture; and wherein the fatty acid oil mixture comprises from about 80% to about 88% eicosapentaenoic acid ethyl ester and docosahexaenoic acid ethyl ester, by weight of the fatty acid oil mixture; from about 0.1% to about 3% surfactant, by weight of the emulsion; from about 0.1% to about 6% CaCl2.2H2O, by weight of the emulsion; and from about 1% to about 15% water, by weight of the emulsion.

The present disclosure is further directed to a capsule comprising a polysaccharide gel membrane outer surface shell comprising at least one alginate wherein: the outer surface encapsulates an emulsion comprising at least one oily phase; the at least one oily phase comprises a fatty acid oil mixture and at least one surfactant; the fatty acid oil mixture comprises at least 75% eicosapentaenoic acid (EPA) and docosahexaenoic acid (DHA), by weight of the fatty acid oil mixture; from about 0.1 to about 3% surfactant, by weight of the emulsion; from about 0.1 to about 6% CaCl2.2H2O, by weight of the emulsion; from about 0.5 to about 5% water, by weight of the emulsion; and the emulsion does not comprise marmelo mucilage.

The present disclosure is further directed to a method of regulating at least one health problem in a subject in need thereof comprising administering to the subject a capsule comprising: a polysaccharide gel membrane outer surface shell comprising at least one alginate wherein: the outer surface encapsulates an emulsion comprising at least one oily phase; the at least one oily phase comprises a fatty acid oil mixture and at least one surfactant; the fatty acid oil mixture comprises at least 75% eicosapentaenoic acid (EPA) and docosahexaenoic acid (DHA), by weight of the fatty acid oil mixture; from about 0.1 to about 3% surfactant, by weight of the emulsion; from about 0.1 to about 6% CaCl2.2H2O, by weight of the emulsion; from about 0.5 to about 5% water, by weight of the emulsion; and the emulsion does not comprise marmelo mucilage; wherein the at least one health problem is chosen from irregular plasma lipid levels, cardiovascular functions, immune functions, visual functions, insulin action, neuronal development, hypertriglyceridemia, heart failure, and post myocardial infarction.

The present disclosure is further directed to a capsule comprising a polysaccharide gel membrane outer surface shell comprising at least one alginate wherein: the outer surface encapsulates an emulsion comprising at least one oily phase; the at least one oily phase comprises a fatty acid oil mixture and at least one surfactant; the fatty acid oil mixture comprises at least 95% eicosapentaenoic acid (EPA), by weight of the fatty acid oil mixture; and the emulsion does not comprise marmelo mucilage.

The present disclosure is further directed to a capsule comprising a polysaccharide gel membrane outer surface shell comprising at least one alginate wherein: the outer surface encapsulates an emulsion comprising at least one oily phase; the at least one oily phase comprises a fatty acid oil mixture and at least one surfactant; the fatty acid oil mixture comprises less than 75% eicosapentaenoic acid (EPA) and docosahexaenoic acid (DHA), by weight of the fatty acid oil mixture; and the emulsion does not comprise marmelo mucilage.

The present disclosure is further directed to an oil-in-water emulsion to be encapsulated comprising: from about 80% to about 85% of at least one fatty acid oil mixture by weight of the emulsion; wherein the fatty acid oil mixture comprises less than 75% eicosapentaenoic acid (EPA) and docosahexaenoic acid (DHA), by weight of the fatty acid oil mixture from about 0.1% to about 3% surfactant, by weight of the emulsion; from about 0.1% to about 6% CaCl2.2H2O, by weight of the emulsion; and from about 1% to about 15% water, by weight of the emulsion.

The present disclosure is further directed to a capsule comprising a polysaccharide gel membrane outer surface shell comprising at least one alginate wherein: the outer surface encapsulates an emulsion comprising at least one oily phase; the at least one oily phase comprises a fatty acid oil mixture and at least one surfactant; the fatty acid oil mixture comprises less than 75% eicosapentaenoic acid (EPA) and docosahexaenoic acid (DHA), by weight of the fatty acid oil mixture; from about 0.1% to about 3% surfactant, by weight of the emulsion; from about 0.1% to about 6% CaCl2.2H2O, by weight of the emulsion; from about 0.1% to about 5% water, by weight of the emulsion; and the emulsion does not comprise marmelo mucilage.

The present disclosure is further directed to a method of regulating at least one health problem in a subject in need thereof comprising administering to the subject a capsule comprising: a polysaccharide gel membrane outer surface shell comprising at least one alginate wherein: the outer surface encapsulates an emulsion comprising at least one oily phase; the at least one oily phase comprises a fatty acid oil mixture and at least one surfactant; the fatty acid oil mixture comprises less than 75% eicosapentaenoic acid (EPA) and docosahexaenoic acid (DHA), by weight of the fatty acid oil mixture; from about 0.1 to about 3% surfactant, by weight of the emulsion; from about 0.1 to about 6% CaCl₂.2H₂O , by weight of the emulsion; from about 0.5 to about 5% water, by weight of the emulsion; and the emulsion does not comprise marmelo mucilage; wherein the at least one health problem is chosen from irregular plasma lipid levels, cardiovascular functions, immune functions, visual functions, insulin action, neuronal development, hypertriglyceridemia, heart failure, and post myocardial infarction.

The present disclosure is further directed to a capsule comprising a polysaccharide gel membrane outer surface shell comprising at least one alginate wherein: the outer surface encapsulates an emulsion comprising at least one oily phase; the at least one oily phase comprises an oil and at least one surfactant; and the emulsion does not comprise marmelo mucilage. Still further, the present disclosure is directed to an oil-in-water emulsion to be encapsulated comprising: from about 80% to about 85% of an oil by weight of the emulsion; from about 0.1% to about 3% surfactant, by weight of the emulsion; from about 0.1% to about 6% CaCl₂.2H₂O, by weight of the emulsion; and from about 1% to about 15% water, by weight of the emulsion. The present disclosure is also directed to a capsule comprising a polysaccharide gel membrane outer surface shell comprising at least one alginate wherein: the outer surface encapsulates an emulsion comprising at least one oily phase; the at least one oily phase comprises oil and at least one surfactant; from about 0.1% to about 3% surfactant, by weight of the emulsion; from about 0.1% to about 6% CaCl₂.2H₂O , by weight of the emulsion; from about 0.1% to about 5% water, by weight of the emulsion; and the emulsion does not comprise marmelo mucilage. In addition, the present disclosure is directed to a method of regulating at least one health problem in a subject in need thereof comprising administering to the subject a capsule comprising: a polysaccharide gel membrane outer surface shell comprising at least one alginate wherein: the outer surface encapsulates an emulsion comprising at least one oily phase; the at least one oily phase comprises an oil and at least one surfactant; from about 0.1 to about 3% surfactant, by weight of the emulsion; from about 0.1 to about 6% CaCl₂.2H₂O , by weight of the emulsion; from about 0.5 to about 5% water, by weight of the emulsion; and the emulsion does not comprise marmelo mucilage; wherein the at least one health problem is chosen from irregular plasma lipid levels, cardiovascular functions, immune functions, visual functions, insulin action, neuronal development, hypertriglyceridemia, heart failure, and post myocardial infarction. The oil may be chosen from an unsaturated oil, a monounsaturated oil, a polyunsaturated oil, and saturated oil. Moreover, a pharmaceutical or nutraceutical agent can be suspended, dispersed or dissolved in the oil. It is also contemplated that those claims embodying an oil encompass elements recited throughout the description of the present disclosure. The present disclosure encompasses a fatty acid mixture and/or an oil

### BRIEF DESCRIPTION OF THE DRAWINGS

FIGS. 1(a) to 1(d) graphically show the average plasma concentration versus time curves of EPA and DHA after single oral dose of Omacor® and compositions of the present disclosure comprising K85EE in male minipigs. Specifically, FIG. 1(a) shows the average EPA plasma concentration after oral dosing of 2 g (2 capsules). FIG. 1(b) shows the average DHA plasma concentration after oral dosing of 2 g (2 capsules). FIG. 1(c) shows the average EPA plasma concentration after oral dosing of 4 g (4 capsules). FIG. 1(d) shows the average DHA plasma concentration after oral dosing of 4 g (4 capsules).

FIG. 2 graphically shows the solubility of EPA and DHA in alginate and gelatin capsules.

### DESCRIPTION

Particular aspects of the disclosure are described in greater detail below. The terms and definitions as used in the present application and as clarified herein are intended to represent the meaning within the present disclosure. The patent and scientific literature referred to herein and referenced above are hereby incorporated by reference. The terms and definitions provided herein control, if in conflict with terms and/or definitions incorporated by reference.

The singular forms “a,” “an,” and “the” include plural reference unless the context dictates otherwise.

As used herein, the term “omega-3 fatty acids” includes natural and synthetic omega-3 fatty acids, as well as pharmaceutically acceptable esters, free acids, triglycerides, derivatives, conjugates (see, e.g., Zaloga et al., U.S. Patent Application Publication No. 2004/0254357, and Horrobin et al., U.S. Pat. No. 6,245,811, each hereby incorporated by reference), precursors, salts, and mixtures thereof. Examples of omega-3 fatty acid oils include, but are not limited to, omega-3 polyunsaturated, long-chain fatty acids such as a eicosapentaenoic acid (EPA), docosahexaenoic acid (DHA), α-linolenic acid (ALA); heneicosapentaenoic acid (HPA); docosapentaenoic acid (DPA); eicosatetraenoic acid; and octadecatetraenoic acid; and esters of omega-3 fatty acids with glycerol such as mono-, di- and triglycerides; and esters of the omega-3 fatty acids and a primary, secondary and/or tertiary alcohol, such as, for example, fatty acid methyl esters and fatty acid ethyl esters. Further for example, omega-3 fatty acid oils are long-chain fatty acids, such as, EPA and DHA, triglycerides (TG) thereof, ethyl esters (EE) thereof, and/or mixtures thereof. The omega-3 fatty acids, their esters, triglycerides, derivatives, conjugates, precursors, salts and/or mixtures thereof can be used in their pure form and/or as a component of an oil, for example, as marine oil (e.g., fish oil and purified fish oil concentrates), microbial oils and plant-based oils.

The fatty acid oil mixture of the present disclosure comprises omega-3 fatty acids, such as EPA and DHA. The oil mixture may further comprise at least one other omega-3 fatty acid other than EPA and DHA chosen from α-linolenic acid, heneicosapentaenoic acid, docosapentaenoic acid, eicosatetraenoic acid, and octadecatetraenoic acid. Examples of further omega-3 fatty acids and mixtures thereof encompassed by the present disclosure include the omega-3 fatty acids defined in the European Pharmacopoeia Omega-3 Ethyl Ester 90 and purified marine oils, for example, as defined in the European Pharmacopoeia Omega-3 Triglycerides, the European Pharmacopoeia Omega-3 acid Ethyl Esters 60, or the Fish oil rich in omega-3 acids mongraph.

Commercial examples of omega-3 fatty acids suitable for the present disclosure comprising different fatty acid mixtures (e.g., that can be in the form of triglycerides (TG), ethyl esters (EE), free fatty acid form (FA) and/or as phospholipids) include, but are not limited to: Incromega™ omega-3 marine oil concentrates such as Incromega™ TG7010 SR, Incromega™ E7010 SR, Incromega™ TG6015, Incromega™ EPA500TG SR, Incromega™ E400200 SR, Incromega™ E4010, Incromega™ DHA700TG SR, Incromega™ DHA700E SR, Incromega™ DHA500TG SR, Incromega™ TG3322 SR, Incromega™ E3322 SR, Incromega™ TG3322, Incromega™ E3322, Incromega™ Trio TG/EE (Croda International PLC, Yorkshire, England); EPAX6000FA, EPAX5000TG, EPAX4510TG, EPAX2050TG, EPAX7010EE, EPAX5500EE, EPAX5500TG, EPAX5000EE, EPAX5000TG, EPAX6000EE, EPAX6000TG, EPAX6000FA, EPAX6500EE, EPAX6500TG, EPAX4510TG, EPAX1050TG, EPAX2050TG, EPAX 7010TG, EPAX7010EE, EPAX6015TG/EE, EPAX4020TG, and EPAX4020EE (EPAX is a wholly-owned subsidiary of Norwegian company Austevoll Seafood ASA); Omacor®/Lovaza™/Zodin®/Seacor® finished pharmaceutical product, K85EE, and AGP 103 (Pronova BioPharma Norge AS); MEG-3® EPA/DHA fish oil concentrates (Ocean Nutrition Canada); DHA FNO “Functional Nutritional Oil” and DHA CL “Clear Liquid” (Lonza); Superba™ Krill Oil (Aker); omega-3 products comprising DHA produced by Martek; Neptune krill oil (Neptune); cod-liver oil products and anti-reflux fish oil concentrate (TG) produced by Mailers; Lysi Omega-3 Fish oil; Seven Seas Triomega® Cod Liver Oil Blend (Seven Seas); Fri Flyt Omega-3 (Vesterålens); and Epadel (Mochida).

Additional oils include triglyceride vegetable oils, commonly known as long chain triglycerides such as castor oil, corn oil, cottonseed oil, olive oil, peanut oil, safflower oil, sesame oil, soybean oil, hydrogenated soybean oil and hydrogenated vegetable oils; medium chain triglycerides such as those derived from coconut oil or palm seed oil, monoglycerides, diglycerides and triglycerides. In addition to mixed glycerides there are other oils such as esters of propylene glycol such as mixed diesters of caprylic/capric acids of propylene glycol, esters of saturated coconut and palm kernel oil-derived caprylic, linoleic, succinic or capric fatty acids glycerin or propylene glycol and esters formed between fatty acids and fatty alcohols such as esters formed between capric or caprylic acid and glycerol. Other oils within the scope of the present invention are those that include naturally occurring emulsifiers. One such oil is soy oil, which contains lecithin. Lecithin is useful in food manufacturing as an emulsifier in products high in fats and oils. Preferred oils within the scope of the present invention are those that are a liquid, or that can be made into a liquid at a temperature in the range of, for example, 20° C. to 95° C.

The fatty acid oil mixture according to the present disclosure may derived from or a component of animal oils or non-animal oils. In some embodiments of the present disclosure, the mixture of omega-3 fatty acids may be from at least one oil chosen from marine oils, plant-based oils, and microbial oils. Marine oils include, for example, fish oil, krill oil, and lipid composition derived from fish. Plant-based oils include, for example, flaxseed oil, canola oil, mustard seed oil, and soybean oil. Microbial oils include, for example, products by Martek. The oil mixture may further comprise at least one omega-6 fatty acid.

In some embodiments of the present disclosure, the fatty acids, such as omega-3 fatty acids, are esterified, such as alkyl esters. Those alkyl esters may include, but are not limited to, ethyl, methyl, propyl, and butyl esters, and mixtures thereof. In at least one embodiment, the omega-3 fatty acids are present in the form of free fatty acids (FA).

According to another embodiment, the fatty acids are chosen from mono-, di-, and triglycerides. In another embodiment, the fatty acids are in the form of a phospholipid.

In some embodiments, the fatty acid oil mixture and/or the oily phase can serve as an active pharmaceutical ingredient (API). In some embodiments, the oil mixture may comprise a flavor oil, a food, and/or a food additive. The oil mixture may also be a carrier for oil-soluble active materials, wherein said oil-soluble active material comprises another pharmaceutical agent, nutritional agent, flavor, fragrance, or a food.

The oil itself can be an active ingredient such as a food or a pharmaceutical, nutraceutical, veterinary active ingredient or it can be a carrier for a food or an active ingredient such as a pharmaceutical, nutraceutical or veterinary active agent. When the oil is used as a carrier for a food or an active ingredient such as a pharmaceutical, nutraceutical or veterinary active agent, the food or an active ingredient such as a pharmaceutical, nutraceutical or veterinary active agent can be dissolved in the oil or dispersed in the oil. The oil may be selected from any oil, or combination of oils, that find utility in an encapsulated form, for example, for use in the pharmaceutical, veterinary, nutraceutical, and food industries. Suitable oils include, without limitation, oils derived from marine and non-marine sources including fish, animals, plants, microorganisms, or extracts thereof; oils that are chemical compounds derived by synthetic or other means, or formulations thereof; or oils that are fatty acids, esters, salts or derivatives thereof.

In at least one embodiment of the present disclosure, the capsules comprise a marine oil, such as a fish oil.

As used herein, the term “alginate” includes alginic acid and/or pharmaceutically acceptable salts thereof, and refers generally to a copolymer comprising (1-4)-linked β-D-mannuronate (M) and its C-5 epimer α-L-guluronate (G) residues. Non-limiting examples of alginate salts suitable for the disclosure herein include alginate salts of calcium, strontium, barium, and aluminum. In one embodiment, alginate comprises all or in part M-alginate. In another embodiment, alginate comprises all or in part G-alginate. In another embodiment, alginate comprises a combination of M-alginate and G-alginate. In at least one embodiment, the alginate has a G-alginate content of at least 30% by weight. In other embodiments, the alginate has a G-alginate content ranging from about 40% to about 80% by weight. In at least one embodiment, the alginate comprises about 1% to about 80%, by weight with respect to the total weight of the shell. In at least one embodiment of the present disclosure, the alginate comprises M-alginate, G-alginate, or a combination thereof. In at least one embodiment, the alginate comprising the outer surface shell of the capsule comprises M-alginate.

In one embodiment, the alginate in the shell is a polyvalent metal ion alginate having: (a) having an M content of from 50%-62% by weight based on the weight of the M and G content; and (b) a viscosity of from 35-80 cps when measured as a monovalent metal ion alginate in a 3.5% water solution at 20° C. using a Brookfield LV viscometer at 60 rpm and spindle #1).

In at least one embodiment, the alginate shell achieves a time-release delivery of omega-3 fatty acids upon administration to a subject.

In some embodiments of the present disclosure, the alginate shell further comprises coloring agents, stabilizers, sweetening agents, plasticizers, and/or hardeners.

In at least one embodiment, the alginate shell comprises from about 10% to about 80% plasticizer by weight with respect to the total shell weight

Other polymers contemplated as comprising the capsule shell include polyesters, polyacrylates, polycyanoacrylates, polysaccharides, polyethylene glycol, and mixtures thereof. Other polymers may include, for example, gelatin, carboxymethylcellulose alginates, carrageenans, pectins, ethyl cellulose, hydroxypropyl methylcellulose, cellulose acetophthalate, hydroxypropyl methylcellulose phthalate, methylacrylicacid copolymers (Eudragit® L and S), dimethylaminoethylmethacrylate copolymers (Eudragit E), trimethylammoniumethylmethacrylate copolymers (e.g., Eudragit® RL and RS), polymers and copolymers of lactic and glycolic acids, and mixtures thereof. In one embodiment, the polymer comprises a plasticizer additive, such as, for example, but not limited to, triethyl citrate, butyl phthalate, and mixtures thereof. Other additives may optionally be incorporated to improve and/or facilitate the encapsulation process, such as, for example, fluidizing agents, such as talc.

The capsules of the present disclosure may comprise at least one non-active pharmaceutical ingredient (also known generally herein as “excipients”). Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. The at least one non-active ingredient may be chosen from colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide, and xanthum gum.

Surfactants may be chosen from, for example, glycerol acetates and acetylated glycerol fatty acid esters, such as acetin, diacetin, triacetin, and/or mixtures thereof. Suitable acetylated glycerol fatty acid esters include, but are not limited to, acetylated monoglycerides, acetylated diglycerides, and mixtures thereof.

In addition, the surfactant may be chosen from glycerol fatty acid esters, such as, for example, those comprising a fatty acid component of about 6-22 carbon atoms. Glycerol fatty acid esters may be chosen from monoglycerides, diglycerides, triglycerides, and/or mixtures thereof. Suitable glycerol fatty acid esters include, but are not limited to, monoglycerides, diglycerides, medium chain triglycerides with fatty acids having about 6-12 carbons, and mixtures thereof. Capmul® MCM (medium chain mono- and di-glycerides) is an example.

Surfactants according to the present disclosure may be chosen from propylene glycol esters. For example, propylene glycol esters include, but are not limited to, propylene carbonate, propylene glycol monoacetate, propylene glycol diacetate, propylene glycol fatty acid esters, acetylated propylene glycol fatty acid esters, propylene glycol fatty acid monoesters, propylene glycol fatty acid diesters, and mixtures thereof. Fatty acids may comprise, for example, about 6-22 carbon atoms. Examples of propylene glycol esters include, but are not limited to, propylene glycol monocaprylate (Capryol®), propylene glycol dicaprylate, propylene glycol dicaprate, propylene glycol dicaprylate/dicaprate, and mixtures thereof.

Surfactants according to the present disclosure may be chosen from ethylene glycol esters, such as, for example, monoethylene glycol monoacetates, diethylene glycol esters, polyethylene glycol esters, and mixtures thereof. Additional examples include ethylene glycol monoacetates, ethylene glycol diacetates, ethylene glycol fatty acid monoesters, ethylene glycol fatty acid diesters, and mixtures thereof. In addition, the ethylene glycol esters may be chosen from polyethylene glycol fatty acid monoesters, polyethylene glycol fatty acid diesters, and mixtures thereof. Ethylene glycol esters may be obtained from the transesterification of polyethylene glycol and a triglyceride, a vegetable oil, and/or mixture thereof, and include, for example, those marketed as Labrafil® and Labrasol®. Polyoxyethylene-sorbitan-fatty acid esters (also called polysorbates, such as polysorbate 20, polysorbate 40, and polysorbate 80), e.g., of from 4 to 25 alkylene moieties, for example monolauryl, trilauryl, palmityl, stearyl, and oleyl esters, including, for example, Tween®, such as Tween® 80, Tween® 40, and Tween® 20. Further examples of surfactants that may be used in the present disclosure include Crillet, such as Crillet 4 and Crillet 1, Span 20, and Crill 1. In at least one embodiment of the present disclosure, the surfactant is chosen from polysorbate 20, polysorbate 40, and polysorbate 80.

Another group of suitable surfactants includes propylene glycol monocaprylate, mixtures of glycerol and polyethylene glycol esters of long fatty acids, polyethoxylated castor oils, nonylphenol ethoxylates (Tergitol®), glycerol esters, oleoyl macrogol glycerides, propylene glycol monolaurate, propylene glycol dicaprylate/dicaprate, polyethylene-polypropylene glycol copolymer, and polyoxyethylene sorbitan monooleate. Further examples include Poloxamer 188, Pluronic/Lutrol F68, Brij 96V, Cremophor EL, Etocas 35 HV, Cremophor RH 40, HCO-40, Croduret 40 LD, Cremophor RH 60, HCO-60, and Solutol HS-15.

Another group of suitable surfactants includes phospholipids, such as soybean lecithin, egg lecithin, diolelyl phosphatidylcholine, distearoyl phosphatidyl glycerol, PEG-ylated phospholipids, and dimyristoyl phosphatidylcholine.

Hydrophilic solvents which may be used include, but are not limited to, alcohols, including water miscible alcohols, such as absolute ethanol and/or glycerol. Other alcohols include glycols, e.g., any glycol obtainable from an oxide such as ethylene oxide, e.g., 1,2-propylene glycol. Other non-limiting examples include polyols, e.g., a polyalkylene glycol, e.g., poly(C₂₋₃)alkylene glycol. One non-limiting example is a polyethylene glycol. The hydrophilic component may comprise an N-alkylpyrollidone, such as, but not; limited to, N—(C₁₋₁₄ alkyl)pyrollidone, e.g., N-methylpyrollidone, tri(C₁₋₄alkyl)citrate, e.g., triethylcitrate, dimethylisosorbide, (C₅₋₁₃) alkanoic acid, e.g., caprylic acid and/or propylene carbonate. The hydrophilic solvent may comprise a main or sole component, e.g., an alcohol, e.g., C₁₋₄-alcohol, e.g., ethanol, or alternatively a cocomponent, e.g., which may be chosen from partial lower ethers or lower alkanols. Suitable partial ethers include, for example, Transcutol® (which has the formula C₂H₅—[O—(CH₂)₂]₂—OH), Glycofurol® (also known as tetrahydrofurfuryl alcohol polyethylene glycol ether), or lower alkanols such as ethanol, such as, for example, glycerol acetates and acetylated glycerol fatty acid esters.

In some embodiments of the present disclosure, the capsules encapsulate at least one oily phase comprising a fatty acid oil mixture water, and at least one surfactant. In some embodiments, the oily phase comprises an emulsion, such as an oil-in-water emulsion, a water-in-oil emulsion, or a water-in-oil-in-water emulsion.

The at least one oily phase may further comprise omega-6 fatty acids. Examples of omega-6 fatty acids include, but are not limited to, linoleic acid, gamma-linoleic acid, and arachidonic acid.

According to some embodiments of the present disclosure, the emulsion comprises at least 30% oil mixture by weight of the emulsion, such as at least 35%, at least 40%, at least 45%, at least 50%, at least 55%, at least 60%, at least 65%, at least 70%, at least 75%, at least 80%, at least 85%, at least 90%, or even at least 95% oil mixture by weight of the emulsion. For example, in some embodiments, the emulsion comprises from about 75% to about 90% oil mixture by weight of the emulsion, such as from about 80% to about 85% oil mixture by weight of the emulsion, 85% to about 90% oil mixture by weight of the emulsion.

In some embodiments, the fatty acid oil mixture comprises at least 70% omega-3 fatty acids by weight of the fatty acid oil mixture, such as at least 75% by weight, at least 80% by weight, at least 90% by weight, or even about 95% by weight of the fatty acid oil mixture. In at least one embodiment, the fatty acid oil mixture is a pharmaceutical oil mixture comprising about 90% to 95% omega-3 fatty acids by weight of the fatty acid oil mixture. In at least one embodiment, the fatty acid oil mixture comprises at least 80% omega-3 fatty acids, by weight of the fatty acid oil mixture.

The fatty acid oil mixture may comprise, for example, EPA, DHA, DPA, HPA, or any combination thereof. The EPA, DHA, DPA, and HPA may be, for example, independently from each other in a form chosen from ethyl ester, free fatty acid, and triglyceride. In at least one embodiment, the fatty acid oil mixture further comprises at least one omega-3 fatty acid other than EPA and DHA chosen from α-linolenic acid, heneicosapentaenoic acid, docosapentaenoic acid, eicosatetraenoic acid, and octadecatetraenoic acid.

In some embodiments, the sum of EPA and DHA comprises greater than 70% by weight of the fatty acid oil mixture, such as greater than 75% by weight, greater then 80% by weight, greater than 85% by weight, greater than 90% by weight, or even greater than 95% by weight of the fatty acid oil mixture. For example, in some embodiments, the sum of EPA and DHA comprises from about 70% to about 95% by weight of the fatty acid oil mixture, such as from about 75% to about 90% by weight, and such as from about 80 to about 88% by weight of the fatty acid oil mixture. In at least one embodiment, the fatty acid oil mixture comprises at least 80% EPA and DHA, by weight of the fatty acid oil mixture. In at least one embodiment, the sum of EPA and DHA comprises about 84% by weight of the fatty acid oil mixture.

In some embodiments of the present disclosure, the weight ratio of EPA:DHA ranges from about 1:10 to 10:1, from about 1:8 to 8:1, from about 1:6 to 6:1, from about 1:5 to 5:1, from about 1:4 to 4:1, from about 1:3 to 3.1, or from about 1.2 to 2:1. In at least one embodiment, the weight ratio of EPA:DHA ranges from about 1:2 to 2:1. In at least one embodiment, the weight ratio of EPA:DHA ranges from about 1:1 to 2:1. In at least one embodiment, the weight ratio of EPA:DHA ranges from about 1.2 to 1.3.

In some embodiments of the present disclosure, the capsule is a pharmaceutical formulation, wherein the sum of EPA and DHA comprises at least 75% by weight of the fatty acid oil mixture, such as 80%, 85%, 90%, 95%, or any number in between, by weight of the fatty acid oil mixture. In some embodiments, for example, the sum of EPA and DHA comprises from about 75% to about 95% by weight of the fatty acid oil mixture, such as from about 75% to about 90% by weight of the fatty acid oil mixture, from about 75% to about 85% by weight of the fatty acid oil mixture, from about 75% to about 80% of the fatty acid oil mixture, from about 80% to about 95% by weight of the fatty acid oil mixture, from about 80% to about 90% by weight of the fatty acid oil mixture, from about 80% to about 85% by weight of the fatty acid oil mixture, from about 85% to about 95% by weight of the fatty acid oil mixture, from about 85% to about 90% by weight of the fatty acid oil mixture, and further for example, from about 90% to about 95% by weight of the fatty acid oil mixture, or any number in between. In at least one embodiment, the sum of EPA and DHA comprises from about 80% to about 85%, such as 84%, by weight of the fatty acid oil mixture.

In some embodiments, the fatty acid oil mixture comprises at least 90% EPA by weight of the fatty acid oil mixture, such as at least 95% EPA by weight of the fatty acid oil mixture. In at least one embodiment, the capsule is a pharmaceutical formulation, wherein the fatty acid oil mixture comprises at least 95% EPA by weight of the fatty acid oil mixture.

In other embodiments, the capsule is a food or a nutritional supplement, wherein the sum of EPA and DHA comprises less than 75% by weight of the fatty acid oil mixture. In some embodiments, for example, the sum of EPA and DHA comprises less than 70%, less than 65%, less than 60%, less than 55%, less than 50%, less than 45%, less than 40%, or even less than 35% by weight of the fatty acid oil mixture. In some embodiments, the sum of EPA and DHA comprises from about 30% to about 75% by weight of the fatty acid oil mixture, such as from about 30% to about 70% by weight of the fatty acid oil mixture, from about 30% to about 65% by weight of the fatty acid oil mixture, from about 30% to about 55% by weight of the fatty acid oil mixture, from about 30% to about 50% by weight of the fatty acid oil mixture, from about 30% to about 45% by weight of the fatty acid oil mixture, from about 30% to about 40% by weight of the fatty acid oil mixture, and further for example, from about 30% to about 35% by weight of the fatty acid oil mixture. For example, in some embodiments, the sum of EPA and DHA comprises 70%, 65%, 60%, 55%, 50%, 45%, 40%, 35%, 30%, or any number in between, by weight of the fatty acid oil mixture. In a further embodiment, the sum of EPA and DHA comprises from about 30% to about 35%, from about 35 to about 40%, from about 40%, to about 45%, from about 45% to about 50%, from about 50% to about 55%, from about 60% to about 65%, from about 65% to about 70% and still further, from about 70% to about 75%, of the fatty acid oil mixture. In at least one embodiment, the EPA and DHA are present in an amount ranging from about 35% to about 75%, by weight of the fatty acid oil mixture, from about 40% to about 70 EPA and DHA, by weight of the fatty acid oil mixture, from about 40% to about 65% EPA and DHA, by weight of the fatty acid oil mixture, from about 40% to about 60% EPA and DHA, by weight of the fatty acid oil mixture, from about 40% to about 55% EPA and DHA, by weight of the fatty acid oil mixture, or from about 50% to about 55% EPA and DHA, by weight of the fatty acid oil mixture. The emulsion may comprise from about 0.05% to about 25% water by weight of the emulsion, such as from about 0.1% to about 20% by weight of the emulsion, such as from about 0.1% to about 15% by weight of the emulsion. The water may be purified. The oil-in-water emulsion to be encapsulated may comprise, for example, from about 0.5% to about 20% water by weight of the emulsion, such as from about 1% to about 15% water by weight of the emulsion, or even from about 1% to about 10% water by weight of the emulsion. In some embodiments, the emulsion after encapsulation comprises from about 0.05% to about 10% water by weight of the emulsion, such as from about 0.1% to about 7% water by weight of the emulsion, or even from about 0.5 to about 5% water by weight of the emulsion.

The emulsion may comprise from about 0.1% to about 5% surfactant by weight of the emulsion, such as from about 0.1% to about 4% by weight of the emulsion, such as from about 0.1% to about 3% by weight of the emulsion.

The emulsion may comprise from about 0.1% to about 10% of at least one gelling agent by weight of said emulsion, such as from about 0.1% to about 8% by weight of the emulsion, such as from about 0.1% to about 6% by weight of the emulsion. In at least one embodiment, the gelling agent is calcium chloride dihydrate (CaCl₂.2H₂O).

The emulsion may further comprise at least one antioxidant. Non-limiting examples of antioxidants in accordance with the present disclosure include α-tocopherol (vitamin E) and calcium disodium EDTA. In at least one embodiment, the emulsion comprises at least one component chosen from anti-oxidants and gelling agents.

In at least one embodiment of the present disclosure, the capsules are seamless. In at least one embodiment, the capsules do not comprise marmelo mucilage.

In at least one embodiment, the capsules comprise a polysaccharide gel membrane outer surface shell, and optionally a coating on said gel membrane. The polysaccharide gel membrane may be ionic. In some embodiments, the polysaccharide gel membrane further comprises one or more secondary film formers. Exemplary secondary film formers include cellulose acetate phthalate, cellulose acetate succinate, methyl cellulose phthalate, ethylhydroxycellulose phthalate, polyvinylacetatephtalate, polyvinylbutyrate acetate, vinyl acetate-maleic anhydride copolymer, styrene-maleic mono-ester copolymer, methyl acrylate-methacrylic acid copolymer, methacrylate-methacrylic acid-octyl acrylate copolymer, propylene glycol alginate, polyvinyl alcohol, carrageenans, pectins, chitosans, guar gum, gum acacia, sodium carboxymethylcellulose, hydroxypropylmethyl cellulose, hydroxypropylcellulose, methylcellulose, starches, and maltodextrins.

In some embodiments of the present disclosure, the polysaccharide gel membrane comprising the seamless capsules is an ionic gel membrane comprising at least one of alginate, propylene glycol alginate, and pectin. Said at least one of alginate, propylene glycol alginate, and pectin may be present in the form of a pharmaceutically-acceptable salt, non-limiting examples of which include salts of calcium, strontium, barium, or aluminum. The ionic polysaccharide of the seamless capsules presently disclosed may comprise an alginate having a weight-average molecular weight ranging from about 20,000 Daltons to about 500,000 Daltons, such as from about 50,000 Daltons to about 500,000 Daltons, or about 100,000 Daltons to about 500,000 Daltons, or about 150,000 Daltons to about 500,000 Daltons, or about 150,000 Daltons to about 300,000 Daltons, or about 20,000 Daltons to about 200,000 Daltons, or from about 20,000 Daltons to about 100,000 Daltons, or from about 30,000 Daltons to about 80,000 Daltons, or from about 30,000 Daltons to about 60,000 Daltons, or even ranging from about 30,000 Daltons to about 40,000 Daltons. In some embodiments of the present disclosure, the ionic polysaccharide comprises a mixture of two alginate components, such as a mixture of (i) an alginate having a weight-average molecular weight ranging from about 30,000 Daltons to about 40,000 Daltons; and (ii) an alginate having a weight-average molecular weight ranging from about 150,000 Daltons to about 500,000 Daltons. In some embodiments, the ratio of (i) to (ii), (i):(ii), may range from about 0.1 to about 20, such as about 1 to about 16.

The capsules presently disclosed may be spherical or in a shape other than spherical. For example, in some embodiments of the present disclosure, the capsules are oblong, oval, or cylindrical. The capsules may be wet or dry.

The thickness of the polysaccharide gel membrane comprising the alginate shell of the capsules presently disclosed may range from about 0.01 millimeter (mm) to about 50 millimeters. The polysaccharide gel film may be wet or dry. In some embodiments, the thickness of the polysaccharide gel film ranges from about 0.3 millimeters to about 4 millimeters. In some embodiments, the thickness of the polysaccharide gel film ranges from about 0.04 millimeters to about 0.5 millimeters. In some embodiments, the thickness of the shell ranges from about 0.01 mm to about 5 mm, such as from about 0.03 mm to about 1 mm, from about 0.05 mm to about 0.5 mm, from about 0.05 mm to about 0.2 mm, from about 0.05 mm to about 0.17 mm, or even from about 0.05 mm to about 0.15 mm.

The capsules according to the present disclosure may have a wet capsule diameter ranging from about 0.5 millimeters to about 50 millimeters, such as about 1 millimeter to about 40 millimeters, wherein the gel membrane has a thickness ranging from about 0.1 millimeter to about 5 millimeters, such as about 0.3 millimeters to about 4 millimeters.

In some embodiments, the capsule is dried, and the gel membrane comprises a dry polysaccharide gel film on the outer surface which constitutes up to 10% by weight of the dried capsule. In some embodiments, the dry capsule has a diameter ranging from about 0.5 millimeters to about 35 millimeters, wherein the dry polysaccharide gel film has a thickness ranging from about 0.01 millimeters to about 5 millimeters. In some embodiments, the thickness of the dry polysaccharide gel film ranges from about 0.04 millimeters to about 0.5 millimeters.

In some embodiments, the capsules comprise from about 0.400 g to about 1.300 g of oil mixture comprising omega-3 fatty acids. For example, in some embodiments, the capsules comprise from about 0.400 g to about 0.800 g of oil mixture, such as from about 0.500 g to about 0.700 g of oil mixture, such as from about 0.600 g to about 0.650 g of oil mixture, or from about 0.500 g to about 0.550 g of oil mixture. In some embodiments, the capsules comprise approximately 0.650 g of oil mixture. In some embodiments, the capsules comprise approximately 0.550 g of oil mixture. In at last one embodiment, the capsules comprise approximately 0.600 g of oil mixture. In other embodiments, the capsules comprise from about 0.800 g to about 1.300 g of oil mixture, such as from about 1.000 g to about 1200 g of oil mixture, such as from about 1.100 g to about 1.250 g of oil mixture. In at least one embodiment, the capsules comprise approximately 1.150 g of oil mixture. In at least one embodiments, the capsules comprise approximately 1.200 g of oil mixture.

According to the present disclosure, the omega-3 fatty acids may be administered to a subject, in need thereof, as capsules with a total capsule weight ranging from about 0.100 g to about 10.000 g, such as about 0.500 g to about 8.000 g, including from about 0.250 g to about 5.000 g and about 0.400 g to about 2.000 g. In the unit dosage form, the capsules comprising omega-3 fatty acids may comprise, for example, a total capsule weight ranging from about 0.100 g to about 4.000 g, such as about 1.000 g to about 4.000 g, further such as 2.000 g and/or 4.000 g unit dosages. In at least one embodiment, the capsules are administered to a subject in a unit dose ranging from about 0.400 g to about 2.000 g, such as about 0.400 g to about 1.740 g, such as about 0.420 g to about 1.680 g.

The daily dosage of omega-3 fatty acids may be administered in from 1 to 10 dosages, such as from 1 to 4 times a day, such as once, twice, three times, or four times per day, and further for example, once, twice or three times per day. The administration may be oral or any other form of administration that provides a dosage of omega-3 fatty acid to a subject.

In one embodiment, the formulation(s) of the present disclosure may allow for improved effectiveness of active ingredients, with one or both administered as a conventional full-strength dose, as compared to the formulations in the prior art. In one embodiment, the formulation(s) of the present disclosure may allow for reduced dosages of omega-3 fatty acids as compared to the formulations in the prior art, while still maintaining or even improving upon the effectiveness of each active ingredient.

According to at least one embodiment, an oil-in-water emulsion is encapsulated in capsules for oral administration, such as seamless capsules. The seamless capsules may also be known generally as softgels.

Seamless capsules of the present disclosure may be prepared, for example, by a method disclosed in WO 2003/084516, comprising: (a) preparing an emulsion comprising oil, water, an emulsifier, and at least one of a water-soluble monovalent metal salt, polyvalent metal salt, and an acid, wherein the oil is present in an amount of at least 50% by weight of the emulsion; and (b) adding at least one portion of the emulsion to an aqueous gelling bath comprised of at least one ionic polysaccharide, thereby encapsulating the at least one portion of the emulsion in a polysaccharide gel membrane, and optionally (c) drying the resulting capsules. The aqueous gelling bath may comprise the alginate in an amount of 3% to 4% by weight of the gelling bath. The gelling bath may also comprise a monovalent metal salt such as sodium chloride in an amount of from 0.1% to 0.5% by weight of the gelling bath. The capsules may then be washed in water before treated in an aqueous plasticizer solution comprising water, glycerol, and a noncrystallizing plasticizer, wherein a weight ratio of the noncrystallizing plasticizer to glycerol is between about 1:1 and about 8:1. The capsules can then be dried.

In one embodiment of the present disclosure, the at least one polyvalent metal salt is calcium chloride (CaCl₂) and the at least one ionic polysaccharide is alginate. In at least one embodiment, the alginate is all or in part M-alginate. In at least one embodiment, the alginate comprises all or in part G-alginate. In at least one embodiment, the alginate comprises a mixture of M-alginate and G-alginate.

An advantage of having an omega 3 fatty acid oil in an alginate capsule, compared to a gelatin capsule, may be the opportunity to include an increased volume of the omega 3 fatty acids as active ingredients because the average film thickness of the seamless alginate capsule is significantly thinner, such as greater than 20% thinner, or greater than 25% thinner, or greater than 30% thinner, or greater than 50% thinner, or greater than 80% thinner, or even greater than 85% thinner, than a gelatin film.

Alginate capsules may offer several benefits over gelatin capsules. For example, alginate capsules may be more temperature-stable and humidity-stable than gelatin capsules. Furthermore, alginate capsules do not require testing for bovine spongiform encephalopathy (SSE) as gelatin capsules do, and alginate capsules may decrease gastrointestinal reflux disease symptoms, such as burping. In addition, alginate capsules may be smaller due to a thinner capsule wall. A thinner wall may allow for increased fill volume for the same capsule size. Increased fill volume may result in a greater dosage per capsule, such that a subject would require fewer capsules per day for a given dose. Alginate capsules may be less sticky, such that they would be easier to swallow and not stick together. The capsules may also be clear and colorless in appearance, which may improve the perception to patients.

Alginate capsules may have an increased fill volume which allows for a larger dosage per unit volume of the capsule. The fill volume of the capsule may increase by about 20%, or about 25%, or even about 30%, in comparison to gelatin capsules. Thus a fewer number of alginate capsules may be administered to a subject in order to achieve the same treatment, such as administration of 3 alginate capsules in place of 4 gelatin capsules. A smaller capsule can also be produced that has the same dosage as a larger gelatin capsule. The smaller size may increase patient compliance in that the capsules can be more easily swallowed. The larger dosage per unit volume of capsule may decrease the number of capsules that would need to be taken to reach a given dose of active pharmaceutical ingredient (API). According to the disclosure herein, API may generally include an oil mixture, such as derived from a marine oil, such as fish oil, krill oil, and lipid compositions derived from fish, plant-based oils, and microbial oils, as well as omega-3 fatty acids comprising the marine oils, plant-based oils, and microbial oils. The capsules presently disclosed may comprise other active pharmaceutical ingredients in addition to marine oils, plant-based oils, and microbial oils. In some embodiments, the capsule may further comprise at least one other active pharmaceutical ingredient microencapsulated in the marine oil or in the capsule shell.

The capsules presently disclosed may be suitable for large dose actives, acid-sensitive actives, actives generating gastric irritation, or oxygen-sensitive actives.

A single alginate capsule of the present disclosure may comprise less or more oil mixture (e.g., API or supplement oil concentrate) than the amount of a gelatin capsule of the same size. For example, the capsules presently disclosed may comprise about 0.5, 0.6, 0.7, 0.8, 0.9, 1.0, 1.1, 1.2, 1.3, 1.4, 1.5, 1.6, 1.7, 1.8, 1.9, or even 2 times the amount of oil mixture as compared to a gelatin capsule of the same size. In at least one embodiment, a single alginate capsule comprises about 0.400 g to about 0.440 g of oil mixture. In another embodiment, a single alginate capsule comprises about 0.800 g to about 0.880 g of oil mixture. In yet another embodiment, a single alginate capsule comprises from about 0.480 g to about 0.520 g of oil mixture. In another embodiment, a single alginate capsule comprises from about 0.980 g to about 1.020 g of oil mixture. In another embodiment, a single alginate capsule comprises from about 1.200 g to about 1.400 g of oil mixture. In another embodiment, a single alginate capsule comprises from about 1.680 g of oil mixture. In another embodiment, a single alginate capsule comprises from about 1.740 g of oil mixture.

The preparation of the capsules, seamless capsules, and/or microcapsules disclosed herein may be carried out following any of the methods described in the literature. By way of description and without being limited thereto, the different processes of obtaining capsules could be grouped into the following categories:

A) Simple Coacervation Method

A solution of the polymer and possible additives of the polymer in a suitable solvent is prepared. The drug to be encapsulated is suspended in said solution and a non-solvent of the polymer is added so as to force the deposit of the polymer on the drug crystals. Examples of such processes can be found in, for example, ES 2009346, EP 0052510, and EP 0346879.

B) Complex Coacervation Method

Complex coacervation method is based on the interaction between two colloids that have opposite electric charges, which generates an insoluble complex that is deposited on the particles of the drug to be encapsulated, forming a membrane that will isolate the drug. Examples of such processes can be found, for example, in GB 1393805.

C) Double Emulsion Method

The drug to be encapsulated is dissolved in water or in a solution of some other coadjuvant and is emulsified in a solution of polymer and additives in a suitable solvent, such as, for example, dichloromethane. The resulting emulsion is in turn emulsified in water or in an aqueous solution of an emulsifying agent, such as polyvinyl alcohol. Once this second emulsion is carried out the solvent in which the polymer and the plasticizer are dissolved is eliminated by means of evaporation or extraction. The resulting microcapsules are obtained directly by filtration or evaporation. Examples of these processes can also be found in patent documents, such as U.S. Pat. No. 4,652,441.

D) Simple Emulsion Method

The drug to be encapsulated, the polymer, and the additives are dissolved together in a suitable solvent. This solution is emulsified in water or in an emulsifier solution, such as polyvinyl alcohol, and the organic solvent is eliminated by evaporation or by extraction. The resulting microcapsules are recovered by filtration or drying. Examples of these processes can also be found, for example, in U.S. Pat. No. 5,445,832.

E) Solvent Evaporation Method

The drug to be encapsulated, the polymer, and additives are dissolved together in a suitable solvent. This solution is evaporated and the resulting residue is micronized to the suitable size. Examples of this process can also be found, for example, in GB 2,209,937.

The above methods may provide for continuous processing and flexibility of batch size. The capsules presently disclosed may be manufactured in low oxygen conditions to inhibit oxidation of the omega-3 fatty acids and/or additional active pharmaceutical ingredients during the manufacturing process.

Capsules according to the present disclosure comprising at least one fatty acid oil mixture may be administered to a subject for therapeutic treatment. The capsules may be administered to a subject in need thereof to regulate at least one health problem, for example, irregular plasma lipid levels, cardiovascular functions, immune functions, visual functions, insulin action, neuronal development, hypertriglyceridemia, heart failure, and post myocardial infarction.

The following examples are intended to illustrate the present disclosure without, however, being limiting in nature. It is understood that the skilled artisan will envision additional embodiments of the invention consistent with the disclosure provided herein.

### WORKING EXAMPLES

### Example 1

### Capsule Preparation

An oil-in-water emulsion was prepared by combining:

Approximately 85% Lovaza™ (about 800-880 mg)

0.2-1.2% Polysorbate 40 by weight

2-6% CaCl₂.2H₂O (gelling salt) by weight

4-15% water by weight

0.01-2 sodium calcium EDTA

The emulsion was extruded through a nozzle and cut into fragments, which were then dropped into a gelling bath. The gelling bath comprised approximately 3.5% by weight of sodium alginate, water and NaCl. The gelling took place for about 20 minutes. The resulting capsules were washed for four hours in purified water and held in an aqueous plasticizer solution comprising water, about 4% by weight of glycerine and 12.5% by weight of a non-crystallizing sorbitol solution (Polysorb-85/70/00). The capsules were then dried.

### Example 2

### Absorption

Bioaccessibility (potential availability for intestinal absorption) of n-3 fatty acids (EPA and DHA) in two alginate compositions (M-alginate and G-alginate) was studied for comparison with a gelatin formulation (Omacor®), where bioaccessibility is defined as the fraction of the compound that is potentially available for intestinal absorption. For lipids, it is assumed that products in the micellar phase are available for absorption.

Experiments were performed under simulated fasting state conditions during transit through a dynamic gastrointestinal model of the stomach and small intestine. During the experiments, samples from different sites of the GI tract were taken in time to provide good insight on the (rate of) digestibility and kinetics of absorption of the nutrients or the stability and activity of functional ingredients.

The following compositions were tested:
(1) K85EE in gelatin capsules (Omacor®); 1000 mg;
(2) K85EE in high M-alginate capsules (“M-alginate” i.e., a polyvalent metal ion alginate having: (a) an M content of from 50%-62% by weight based on the weight of the M and G content; and (b) a viscosity of from 35-80 cps when measured as a monovalent metal ion alginate (e.g., sodium alginate) in a 3.5% water solution at 20° C. using a Brookfield LV viscometer at 60 rpm and spindle #1); 1000 mg;
(3) K85EE in high G-alginate capsules (“G-alginate”); 1000 mg.

Omacor® (composition 1) was commercially-available, and compositions (2) and (3) were prepared according to Example 1. The study was performed in a dynamic, multi-compartmental system of the stomach and small intestine simulating the successive dynamic conditions in the gastric-small-intestinal tract, such as body temperature, pH curves, concentrations of electrolytes, and activity of enzymes in the stomach and small intestine, concentrations of bile salts in the different parts of the gut (for the production of micelles), and kinetics of transit of the GI contents through the stomach and small intestine.

Experiments were performed under simulation of average physiological conditions in the upper gastrointestinal tract of healthy human adults during the fed state and the fasting state conditions. These conditions included especially the dynamics of gastric emptying and intestinal transit times, the gastric and the intestinal pH values, and the composition and activity of the secretion products. Bioaccessibility was expressed as percentage of the intake (2 capsules).

A specific filtration system was used to remove products of lipid digestion and lipophilic compounds that are incorporated in micelles. The formed micelles were filtrated continuously from the jejunum and ileum compartments of the model via hollow fiber semi-permeable membrane systems. The removed material was collected to determine the bio-accessible fraction of fatty acids, cholesterol and fat soluble nutrients/compounds.

Under the fasted state conditions, the release and bioaccessibility of EPA and DHA from all three types of capsules was low. Bioaccessibility in the M-alginate and gelatine capsulates was approximately 2-3% of intake. Bioaccessibility was increased under fed-state conditions with a meal; about 20% for the M-alginate capsules, and about 35% for the gelatin capsules. When corrected for the amount of EPA and DHA delivered into the jejunum (percentage of duodenal delivery), a higher bioaccessibility of EPA and DHA was found in the M-aglinate capsule in comparison to the gelatine capsule.

The M-alginate capsules did not open at the same time in the simulations (without phosphate) as in a phosphate buffer. For G-alginate capsules, EPA and DHA did not release to become bioaccessible during passage through the upper GI tract under fast or fed-state conditions. In the GI tract, phosphate mainly derives from the meal, with small amounts coming from the pancreas and bile secretion.

### Example 3

### Single-Dose Pharmacokinetics

Bioavailability of compositions according to the present disclosure was studied in an animal model (minipig; 5-6 months old) representative of the human digestive system. The animals were orally dosed at two dose levels: 2 g (=2 capsules; “low dose”) and 4 g (=4 capsules; “high dose”). First all animals received 2 g of Omacor®, followed in the next week by 2 g of K85EE alginate capsules (composition 2 as described in Examples 1 and 2). This was subsequently repeated for the high dose groups (4 g) in the third and fourth week. Blood collection took place at pre-dose, 1, 2, 4, 6, 8, 10, 12, 16, 24, and 36 weeks after dosing.

In each plasma sample the EPA and DHA concentrations were determined, as well as cholesterol, triglycerides and HDL levels. An additional set of parameters were determined at pre-dose and 24 h after dosing in the high dose groups; i.e., platelet count (Plt), alanine aminotransferase (ALAT), aspartate aminotransferae (ASAT), bilirubin (Tbil), prothrombine time (PTT), fibrogen (Fib), and activated partial thromboplastine time (APTT). Pharmacokinetic analysis was performed for EPA and DHA, where the data allowed the following parameters were calculated: maximum reached plasma concentration (Cₘₐₓ), time to reach the maximum concentration after dosing (Tₘₐₓ), the terminal half-life (T_{1/2}), the volume of distribution (V_{z}), the total clearance (Cl_{T}), the area under the concentration-time curve extrapolated to infinity (AUC_{0-∞}) and the area under the concentration-time curve extrapolated to the last measured time period (AUC₀₋ₜₙ).

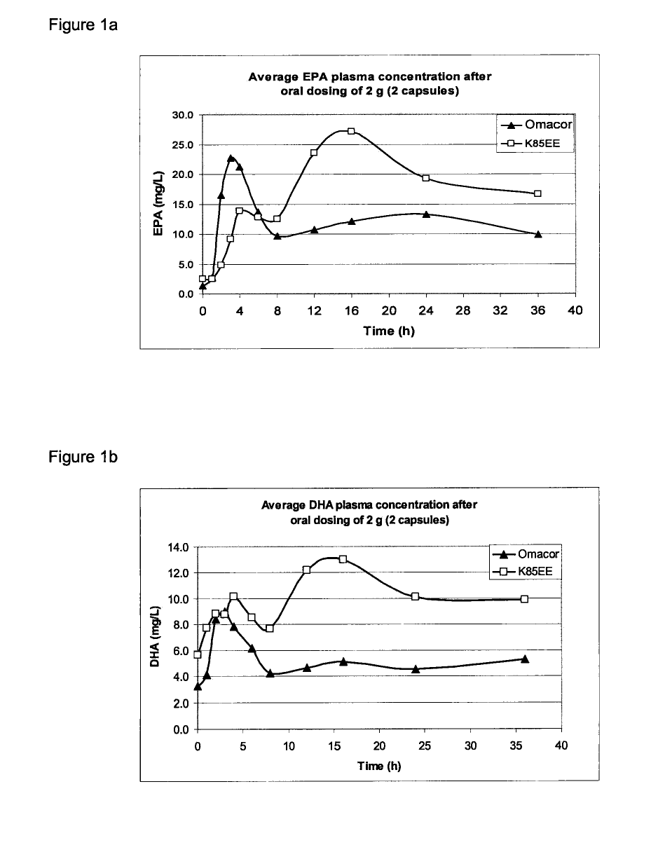

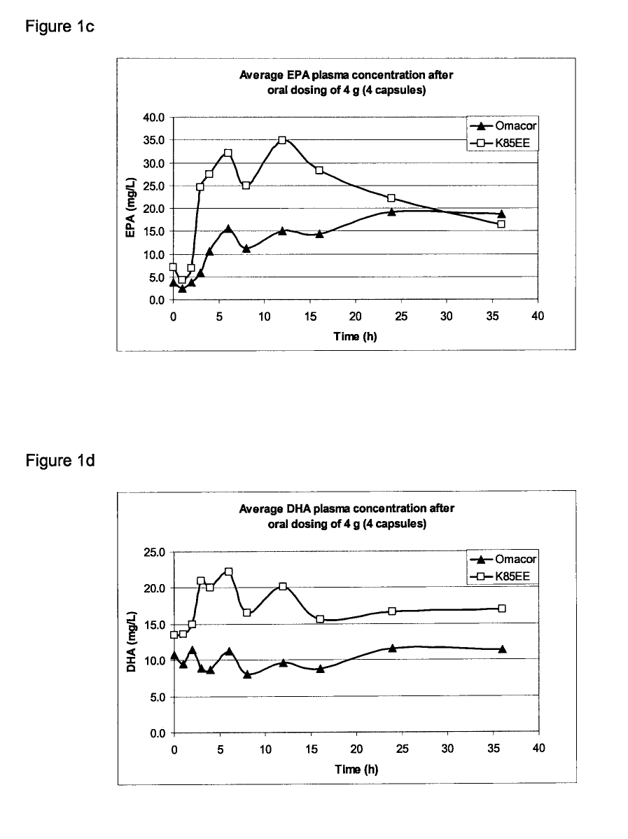

In the low dose group, the K85EE alginate capsules showed a higher uptake of EPA and DHA in comparison with Omacor®. See e.g., FIGS. 1(a) and 1(b). For EPA, the Cₘₐₓ of the K85EE alginate capsules was 27.7 mg/L, and for Omacor®, 22.3 mg/L. The Tₘₐₓ was observed later for the K85EE alginate capsules than for Omacor®, i.e., 21 hours versus 9.5 h, respectively. For DHA, the Cₘₐₓ of the K85EE alginate capsules was 18.6 mg/L, and for Omacor® 14.1 mg/L. The Tₘₐₓ between both formulations was similar (6.5 h). The AUC₀₋ₜₘ for K85EE alginate was on average found to be 1.6 times higher for EPA in comparison with Omacor® and 1.9 times higher for DHA.

The high dose group also showed a higher uptake with the K85EE alginate capsules of EPA and DHA in comparison with Omacor®. See e.g., FIGS. 1(c) and 1(d). For EPA, Cₘₐₓ of the K85EE alginate capsules was 71.7 mg/L, and for Omacor® 25.53 mg/L. The Tₘₐₓ was earlier for the K85EE alginate capsules than for Omacor®, i.e., 11.5 hours versus 23 h, respectively. For DHA, the Cₘₐₓ of the K85EE alginate capsules was 42.4 mg/L and for Omacor® 17.5 mg/L. The Tₘₐₓ for the K85EE alginate capsules was 4.5 h versus 17.5 h for Omacor®. The AUC₀₋ₜₙ for K85EE alginate was on average found to be 1.5 times higher for EPA in comparison with Omacor® and 1.7 times higher for DHA. Results appear in FIGS. 1(a)-(d).

No statistical difference of the following parameters: Cₘₐₓ, Tₘₐₓ, AUC₀₋ₜₙ, AUC_{0-∞}, and T_{1/2} was found between the high and low dose groups due the high variability between the animals within each dose group. After dosing of Omacor® and the K85EE alginate capsules, a decline was seen in all dose groups in the amount of cholesterol and HDL in plasma. The difference in triglycerides concentrations was less prominent.

The K85EE alginate capsules showed higher bioavailability than Omacor® in both dose groups. With 2 g, the bioavailability of EPA was around 1.6 times higher and, for DHA, 1.9 times higher in comparison with Omacor®. If calculated on the geometrical means of the AUCs, the relative bioavailability of K85EE Alginate capsules was even higher, i.e., 2.5 times for both EPA and DHA in comparison with Omacor®. With an oral dose of 4 g, the bioavailability of EPA was 1.5 times higher and for DHA 1.7 times higher in comparison with Omacor®.

The present data support an enhanced bioavailability of EPA and DHA from the K85EE alginate capsules as compared to Omacor®.

### Example 4

### Unit Dose Administration

Examples of oil mixture compositions to be encapsulated:

| | | | High omega-3 | | |
|---|---|---|---|---|---|
| | K85EE/AGP103 | | concentrate | oil supplement | |
| Oil | 100% | by weight | 100% by weight | 100% | by weight |
| mixture | | | | | |
| EPA + | 80-88% | by weight | >75% by weight | <75% | by weight |
| DHA | | | | | |
| EE | | | | | |
| Total | >90% | by weight % | >80% by weight | 35-80% | by weight |
| omega- | | | | | |
| 3 EE | | | | | |

| | Pharmaceutical | High omega-3 | |
|---|---|---|---|
| | oil | concentrate | oil supplement |
| Oil mixture | 100% by weight | 100% by weight | 100% by weight |
| EE, TG or | | | |
| FA form | | | |
| EPA and/or | At least 80% by | >75% by weight | <75% by weight |
| DHA, or | weight | | |
| combination | | | |
| thereof in | | | |
| EE, TG or | | | |
| FA form | | | |
| Total | >90% by weight | >80% by weight | 35-80% by weight |
| omega-3 | | | |
| EE, TG | | | |
| or FA form | | | |

Seamless alginate capsules were prepared according to the procedure of Example 1 for administration to a subject. The capsules were prepared in different unit dosages as shown in Table 1.

TABLE 1
Prepared alginate capsule batches (Examples 4(a)-4(e))

| | Capsule Example | | | | |
|---|---|---|---|---|---|
| | 4(a) | 4(b) | 4(c) | 4(d) | 4(e) |
| Fill content: | 0.644 | 1.130 | 0.544 | 1.234 | 1243 |
| Fatty acid oil mixture | | | | | |
| (g) and surfactant | | | | | |

Further examples of calculated capsules are described in Examples 4(f)-4(i) and shown in Table 2.

### Example 4(f)

The active pharmaceutical ingredient (“API”) was a fatty acid oil mixture (K85EE or AGP103) comprising EPA and DHA present in ester form. The capsule comprised 0.504 g of EPA+DHA, with a total oil mixture weight of 0.600 g, and a total capsule weight of 0.720 g. The capsule comprised about 0.6 times the amount of EPA+DHA of a comparative gelatin capsule (see Table 2).

### Example 4(g)

The active pharmaceutical ingredient was a fatty acid oil mixture (K85EE or AGP103) comprising EPA and DHA present in ester form. The capsule comprised 0.840 g of EPA+DHA, with a total oil mixture weight of 1.000 g, and a total capsule weight of 1.150 g. Thus, the capsule comprised about the same amount (about 1 time the amount) of EPA+DHA of a comparative gelatin capsule.

### Example 4(h)

The active pharmaceutical ingredient was a fatty acid oil mixture (K85EE or AGP103) comprising EPA and DHA present in ester form. The capsule comprised 0.420 g of EPA+DHA, with a total oil mixture of 0.500 g, and a total capsule weight of 0.600 g. Thus, the capsule comprised about 0.5 times the amount of EPA+DHA of a comparative gelatin capsule.

### Example 4(i)

The active pharmaceutical ingredient was a fatty acid oil mixture (K85EE or AGP103) comprising EPA and DHA present in ester form. The capsule comprised 1.008 g of EPA+DHA, with a total oil mixture of 1.200 g, and a total capsule weight of 1.380 g. Thus, the capsule comprised about 1.2 times the amount of EPA+DHA of a comparative gelatin capsule.

TABLE 2
Alginate capsules 4(f)-(i) and comparative gelatin capsule

| | Capsule Example | | | | |
|---|---|---|---|---|---|
| | 4(f) | 4(g) | 4(h) | 4(i) | Gelatin |
| EPA + DHA (g) | 0.504 | 0.840 | 0.420 | 1.008 | 0.840 |
| Fatty acid oil mixture (g) | 0.600 | 1.000 | 0.500 | 1.200 | 1.000 |
| Total capsule weight (g) | 0.720 | 1.150 | 0.600 | 1.380 | 1.430 |

### Example 5a)

### Alginate capsules

Example of alginate capsules:

| | | |
|---|---|---|
| Mixed fatty acid oil (TG, EE or FA form) | 1000 | mg |
| EPA + DHA (TG, EE or FA form) | 840 | mg |
| Alpha-tocopherol | 1-6 | mg |
| Polysorbate 20, 40 or 80 | 4-250 | mg |
| Capsule shell components: | 110-250 | mg |

Example 5b)

### K85EE (or AGP103) Alginate Capsules

Example of alginate capsules:

| | | | | |
|---|---|---|---|---|
| K85EE drug substance | 1000 | mg | 500 | mg |
| EPA + DHA EE | 840 | mg | 420 | mg |
| Alpha-tocopherol | 4 | mg | 2 | mg |
| Polysorbate 40 | 7 | mg | 3.6 | mg |
| Capsule shell components: | Approximately 148 | mg | Approx. 100 | mg |
| Total capsule weight | 1150 | mg | 600 | mg |

It shall be understood that same % relationship between the surfactant and the amount of oil mixture presented in the table above can be used to design other dosage forms.

### Example 6

### Solubility

The solubility of EPA and DHA in alginate and gelatin capsules was tested as follows.

Methods

Alginate capsules: (Batch No. 080520-1) containing KE-85 EE of which 375 mg is docosahexaenoic EE (DHA-EE) and 463 mg is eicosapentaenoic EE (EPA-EE).

Omacor capsules (batch no 6923441) containing KE-85 EE of which 375 mg is docosahexaenoic EE (DHA-EE) and 463 mg is eicosapentaenoic EE (EPA-EE).

Bile salts: Porcine Bile extract, Sigma B8631 lot 037K0196: Contains glycine and taurine conjugates of hyodeoxycholic acid and other bile salts.

Lechitin: Phospholipids, LIPOID S PC from LIPOID AG

Trizma maleate, Sigma Aldrich, T 3128

Oleic acid; Fluka 75096, lot 1333648 51107P25

Monolein; Rylo MG19 Pharma, batch 4010380689, from Danisco

Apparatus: LC Agilent Technologies 1200 series

Column: EclipseXDB C18, 2.1×150 mm, 5 μm, Agilent

Column temperature: 30° C.

Mobile Phase: A: water (0.1%acetic acid), B: MeCN (0.1% acetic acid)

Gradient: 0 to 8 min, from 70%B to 100%B, 8 to 15 minutes: 100% B, from 16 to 16 minutes: from 100% B to 70% B, 16 to 20 minutes: 70% B.

Flow rate: 0.5 ml/min

UV @ 210 nM

Injection volume: 5 μl

Run time: 20 minutes.

Solubilisation in FED State medium

The purpose of the study was to compare the lag time to disintegration of Alginate capsule formulations of KE85-EE in fed state media after pre-treatment of the different capsules in fasted state media for 1 hour. Furthermore the solubilisation rate for KE85-EE of the different formulations in fed state media was followed by HPLC analyses of samples from the micellar phase.

The dissolution experiment was performed with standard dissolution equipment (Erweka DT70, USP 2)

The initial composition of the fasted state media is given in table 1 and the final composition after mixing the solubilisation media is given in table 3.

All the experiments were performed with the following settings: Stirring rate: 200 RPM, Temperature: 37.5° C., Final volume of fasted state media: 100 ml and final volume of fed state media: 500 ml.

Dose in individual dissolution vessels: One capsule of alginate or gelatine containing KE 85-EE (final concentration “total” 1676 μg/ml) was used for the solubilisation study.

TABLE 3
Composition of the media: media 1

| | |
|---|---|
| Bile salts, Porcine (mM) | 0.08 |
| Lechitin (mM) | 0.02 |
| Sodium chloride (mM) | 34.2 |
| Pepsin (mg/ml) | 0.1 |
| pH | 1.6 (adjust with 1M HCl) |
| Osmolarity (mOsm/kg) | 120 |

TABLE 4
Fed State Media

| Lechitin | Oleic | Monoolein | Tris | Sodium | Bile ex- | Ca |
|---|---|---|---|---|---|---|
| mM | acid mM | mM | maleate mM | Chloride mM | tract mM | mM |
| 6.25 | 12 | 6.25 | 12 | 59.5 | 25 | 3.75 |

TABLE 5
Final Composition of Solubilisation Media

| Lechitin | Oleic | Monoolein | Tris | Sodium | Bile ex- | Ca |
|---|---|---|---|---|---|---|
| mM | acid mM | mM | maleate mM | Chloride mM | tract mM | mM |
| 5 | 10 | 5 | 10 | 59.5 | 20 | 3 |

Dissolution Experiment, with Different Capsules

Alginate: To dissolution media 1 (100 ml), equilibrated to 37.5° C. for 30 minutes and stirred at 100 RPM, one alginate capsule containing K85-EE was added. The alginate capsule was stirred for 1 hour in media 1. After 60 minutes media 1 (100 ml containing the capsule) (t=0) was added to media 2 (400 ml) pre-equilibrated to 37° C. for 90 minutes. The pH of the mixed media was adjusted to pH 6.5 with 1 N sodium hydroxide as fast as possible after mixing of the two media. The disintegration of the capsule was followed by visual inspection and the time to disintegration was noted. Samples (2 ml) were withdrawn at the following time points after disintegration of the capsules: 10, 30, 60, 90, 120, 150, 225 min. Immediately after sampling the samples were ultra centrifuged and the concentration of K85-EE8 in the micellar phase was determined by HPLC. Samples from the micellar phase were diluted with acetonitril 1:2 and centrifuged at 10000 rpm for 7 minutes before analyses. N=6.

Gelatine: Dissolution media 1(100 ml), equilibrated to 37.5° C. was stirred at 100 RPM for 1 hour. After 1 hour media 1 (100 ml) was added to media 2 (400 ml) pre-equilibrated to 37° C. for 1 hour. The pH of the mixed media was adjusted to pH 6.5 with 1 N sodium hydroxide as fast as possible after mixing of the two media. 7 mg polysorbate-40(Tween-40) was added to 3 of the vessels. After stirring the mixture for 1 hour one gelatine capsule (t=0) was added to each of the vessels. The disintegration of the capsule was followed by visual inspection and the time to disintegration is noted. Samples (2 ml) were withdrawn at the following time points after disintegration of the capsules: 15, 60, 120, 180 min.

Results:

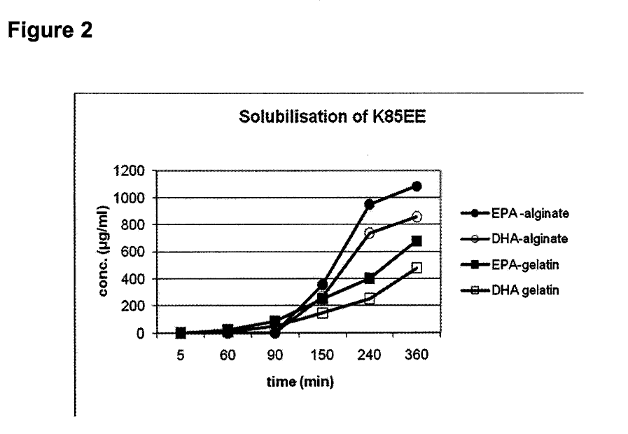

The solubilisation curve for the gelatine capsules is shown in FIG. 2. From the visual inspection it is clear that all the capsules were disintegrated in less than two minutes after being added to the fed state medium. The solubilisation curves for the alginate capsules are shown in figure. From the visual inspection it is clear that all the capsules had disintegrated in less than 70 minutes after being added to the fed state medium.

Comparison of the Different Formulations of KE85-EE:

The individual formulations evaluated under exact same experimental conditions have been compared and the summary data are depicted in figure. From the graphs it is obvious that alginate capsules behaves differently compared to gelatine capsules in the solubilisation process after disintegration of the capsules. The rate of solubilisation for KE85-EE in formulations with alginate is much faster than the rate of solubilisation of KE85-EE in gelatine formulations. The time for solubilisation of KE85-EE from alginate capsules high M will be reached faster than the time to solubilisation of KE85-EE for gelatine capsules, although the time to disintegration is longer for alginate high M than for gelatine capsules.

Other than in the examples, or where otherwise indicated, all numbers expressing quantities of ingredients, reaction conditions, analytical measurements, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

1. A capsule comprising a polysaccharide gel membrane outer surface shell comprising at least one alginate wherein:
the outer surface encapsulates an emulsion comprising at least one oily phase;
the at least one oily phase comprises a fatty acid oil mixture and at least one surfactant;
the fatty acid oil mixture comprises at least 75% eicosapentaenoic acid (EPA) and docosahexaenoic acid (DHA), by weight of the fatty acid oil mixture; and
the emulsion does not comprise marmelo mucilage.

2. The capsule according to claim 1, wherein the EPA and DHA are in a form chosen from ethyl ester, free fatty acid, and triglyceride.

3. The capsule according to claim 1, wherein the fatty acid oil mixture is from at least one oil chosen from marine oil, plant-based oil, and microbial oil.

4. The capsule according to claim 1, wherein the fatty acid oil mixture comprises at least 80% omega-3 fatty acids, by weight of the fatty acid oil mixture.

5. The capsule according to claim 1, wherein the fatty acid oil mixture further comprises at least one omega-3 fatty acid other than EPA and DHA chosen from α-linolenic acid, heneicosapentaenoic acid, docosapentaenoic acid, eicosatetraenoic acid, and octadecatetraenoic acid, wherein the at least one omega-3 fatty acid other than EPA and DHA is in a form chosen from ethyl ester, free fatty acid, and triglyceride.

6. (canceled)

7. The capsule according to claim 1, wherein the at least one oily phase further comprises omega-6 fatty acids and the emulsion further comprises at least one component chosen from anti-oxidants and gelling agents.

8. The capsule according to claim 1, wherein the fatty acid oil mixture comprises at least 80% EPA and DHA, by weight of the fatty acid oil mixture.

9. (canceled)

10. The capsule according to claim 1, wherein the surfactant is chosen from glycerol acetates, glycerol fatty acid esters, acetylated glycerol fatty acid esters, propylene glycol esters, ethylene glycol esters, propylene glycol monocaprylate, mixtures of glycerol and polyethylene glycol esters of long fatty acids, polyethoxylated castor oils, nonylphenol ethoxylates, oleoyl macrogol glycerides, propylene glycol monolaurate, propylene glycol dicaprylate/dicaprate, polyethylene-polypropylene glycol copolymer, polyoxyethylene-sorbitan-fatty acid esters, polyoxyethylene sorbitan monooleate, polysorbate 20, polysorbate 40, polysorbate 80, and phospholipids.

11. (canceled)

12. The capsule according to claim 1, wherein the alginate comprises M-alginate, G-alginate, or a combination thereof.

13. The capsule according to claim 1, wherein the alginate comprises about 1% to about 80%, by weight with respect to the total weight of the shell.

14. The capsule according to claim 1, wherein the shell further comprises at least one additive chosen from coloring agents, stabilizers, sweetening agents, plasticizers, and hardeners.

15. The capsule according to claim 14, wherein the shell comprises from about 10% to about 80% plasticizer by weight with respect to the total shell weight.

16. The capsule according to claim 1, wherein the thickness of the shell ranges from about 0.01 mm to about 5 mm.

17-20. (canceled)

21. The capsule according to claim 1, wherein the fatty acid oil mixture is present in an amount ranging from about 0.400 g to about 1.300 g.

22-27. (canceled)

28. The capsule according to claim 1, wherein the capsule is seamless.

29. The capsule according to claim 1, wherein the capsule is a pharmaceutical formulation.

30. The capsule according to claim 1, wherein the EPA:DHA weight ratio ranges from 1:2 to 2:1.

31. (canceled)

32. (canceled)

33. An oil-in-water emulsion to be encapsulated comprising:
from about 80% to about 85% of at least one fatty acid oil mixture by weight of the emulsion;
wherein the fatty acid oil mixture comprises at least 90% omega-3 ethyl ester fatty acids, by weight of the fatty acid oil mixture; and
wherein the fatty acid oil mixture comprises from about 80% to about 88% eicosapentaenoic acid ethyl ester and docosahexaenoic acid ethyl ester, by weight of the fatty acid oil mixture;
from about 0.1% to about 3% surfactant, by weight of the emulsion;
from about 0.1% to about 6% CaCl₂.2H₂O, by weight of the emulsion; and
from about 1% to about 15% water, by weight of the emulsion.

34. (canceled)

35. (canceled)

36. A capsule comprising a polysaccharide gel membrane outer surface shell comprising at least one alginate wherein:
the outer surface encapsulates an emulsion comprising at least one oily phase;
the at least one oily phase comprises a fatty acid oil mixture and at least one surfactant;
the fatty acid oil mixture comprises at least 75% eicosapentaenoic acid (EPA) and docosahexaenoic acid (DHA), by weight of the fatty acid oil mixture;
from about 0.1 to about 3% surfactant, by weight of the emulsion;
from about 0.1 to about 6% CaCl₂.2H₂O , by weight of the emulsion;
from about 0.5 to about 5% water, by weight of the emulsion; and
the emulsion does not comprise marmelo mucilage.

37. (canceled)

38. (canceled)

39. A method of regulating at least one health problem in a subject in need thereof comprising administering to the subject a capsule comprising:
a polysaccharide gel membrane outer surface shell comprising at least one alginate wherein:
the outer surface encapsulates an emulsion comprising at least one oily phase;
the at least one oily phase comprises a fatty acid oil mixture and at least one surfactant;
the fatty acid oil mixture comprises at least 75% eicosapentaenoic acid (EPA) and docosahexaenoic acid (DHA), by weight of the fatty acid oil mixture;
from about 0.1 to about 3% surfactant, by weight of the emulsion;
from about 0.1 to about 6% CaCl₂.2H₂O , by weight of the emulsion;
from about 0.5 to about 5% water, by weight of the emulsion; and
the emulsion does not comprise marmelo mucilage;
wherein the at least one health problem is chosen from irregular plasma lipid levels, cardiovascular functions, immune functions, visual functions, insulin action, neuronal development, hypertriglyceridemia, heart failure, and post myocardial infarction.

40-61. (canceled)

62. A capsule comprising a polysaccharide gel membrane outer surface shell comprising at least one alginate wherein:
the outer surface encapsulates an emulsion comprising at least one oily phase;
the at least one oily phase comprises a fatty acid oil mixture and at least one surfactant;
the fatty acid oil mixture comprises at least 95% eicosapentaenoic acid (EPA), by weight of the fatty acid oil mixture; and
the emulsion does not comprise marmelo mucilage.

63. A capsule comprising a polysaccharide gel membrane outer surface shell comprising at least one alginate wherein:
the outer surface encapsulates an emulsion comprising at least one oily phase;
the at least one oily phase comprises a fatty acid oil mixture and at least one surfactant;
the fatty acid oil mixture comprises less than 75% eicosapentaenoic acid (EPA) and docosahexaenoic acid (DHA), by weight of the fatty acid oil mixture; and
the emulsion does not comprise marmelo mucilage.

64-93. (canceled)

94. An oil-in-water emulsion to be encapsulated comprising:
from about 80% to about 85% of at least one fatty acid oil mixture by weight of the emulsion;
wherein the fatty acid oil mixture comprises less than 75% eicosapentaenoic acid (EPA) and docosahexaenoic acid (DHA), by weight of the fatty acid oil mixture;
from about 0.1% to about 3% surfactant, by weight of the emulsion;
from about 0.1% to about 6% CaCl₂.2H₂O, by weight of the emulsion; and
from about 1% to about 15% water, by weight of the emulsion.

95. (canceled)

96. (canceled)

97. A capsule comprising a polysaccharide gel membrane outer surface shell comprising at least one alginate wherein:
the outer surface encapsulates an emulsion comprising at least one oily phase;
the at least one oily phase comprises a fatty acid oil mixture and at least one surfactant;
the fatty acid oil mixture comprises less than 75% eicosapentaenoic acid (EPA) and docosahexaenoic acid (DHA), by weight of the fatty acid oil mixture;
from about 0.1% to about 3% surfactant, by weight of the emulsion;
from about 0.1% to about 6% CaCl₂.2H₂O , by weight of the emulsion;
from about 0.1% to about 5% water, by weight of the emulsion; and
the emulsion does not comprise marmelo mucilage.

98. (canceled)

99. (canceled)

100. A method of regulating at least one health problem in a subject in need thereof comprising administering to the subject a capsule comprising:
a polysaccharide gel membrane outer surface shell comprising at least one alginate wherein:
the outer surface encapsulates an emulsion comprising at least one oily phase;
the at least one oily phase comprises a fatty acid oil mixture and at least one surfactant;
the fatty acid oil mixture comprises less than 75% eicosapentaenoic acid (EPA) and docosahexaenoic acid (DHA), by weight of the fatty acid oil mixture;
from about 0.1 to about 3% surfactant, by weight of the emulsion;
from about 0.1 to about 6% CaCl₂.2H₂O, by weight of the emulsion;
from about 0.5 to about 5% water, by weight of the emulsion; and
the emulsion does not comprise marmelo mucilage;
wherein the at least one health problem is chosen from irregular plasma lipid levels, cardiovascular functions, immune functions, visual functions, insulin action, neuronal development, hypertriglyceridemia, heart failure, and post myocardial infarction.

101-126. (canceled)

127. A capsule comprising a polysaccharide gel membrane outer surface shell comprising at least one alginate wherein:
the outer surface encapsulates an emulsion comprising at least one oily phase;
the at least one oily phase comprises an oil and at least one surfactant; and
the emulsion does not comprise marmelo mucilage.

128. An oil-in-water emulsion to be encapsulated comprising:
from about 80% to about 85% of an oil by weight of the emulsion;
from about 0.1% to about 3% surfactant, by weight of the emulsion;
from about 0.1% to about 6% CaCl₂.2H₂O, by weight of the emulsion; and
from about 1% to about 15% water, by weight of the emulsion.

129. A capsule comprising a polysaccharide gel membrane outer surface shell comprising at least one alginate wherein:
the outer surface encapsulates an emulsion comprising at least one oily phase;
the at least one oily phase comprises oil and at least one surfactant;
from about 0.1% to about 3% surfactant, by weight of the emulsion;
from about 0.1% to about 6% CaCl₂.2H₂O , by weight of the emulsion;
from about 0.1% to about 5% water, by weight of the emulsion; and
the emulsion does not comprise marmelo mucilage.

130. A method of regulating at least one health problem in a subject in need thereof comprising administering to the subject a capsule comprising:
a polysaccharide gel membrane outer surface shell comprising at least one alginate wherein:
the outer surface encapsulates an emulsion comprising at least one oily phase;
the at least one oily phase comprises an oil and at least one surfactant;
from about 0.1 to about 3% surfactant, by weight of the emulsion;
from about 0.1 to about 6% CaCl₂.2H₂O , by weight of the emulsion;
from about 0.5 to about 5% water, by weight of the emulsion; and
the emulsion does not comprise marmelo mucilage;
wherein the at least one health problem is chosen from irregular plasma lipid levels, cardiovascular functions, immune functions, visual functions, insulin action, neuronal development, hypertriglyceridemia, heart failure, and post myocardial infarction.

131. (canceled)

132. (canceled)
